(19) United States
(12) Patent Application Publication
(10) Pub. No.: US 2011/0098368 A1
(43) Pub. Date: Apr. 28, 2011

(54) CONTROLLING SYNTHESIS OF METAL SULFIDE CATALYSTS

(75) Inventor: Madhu Anand, Bartlesville, OK (US)

(73) Assignee: ConocoPhillips Company, Houston, TX (US)

(21) Appl. No.: 12/907,137

(22) Filed: Oct. 19, 2010

Related U.S. Application Data
(60) Provisional application No. 61/255,763, filed on Oct. 28, 2009.

(51) Int. Cl. C07C 27/08; C01B 17/20; B01J 27/051

(52) U.S. Cl. 518/714; 423/561.1; 502/220

(57) ABSTRACT

Methods and apparatus relate to catalysts and preparation of the catalysts, which are defined by sulfides of a transition metal, such as one of molybdenum, tungsten, and vanadium. Precipitation forms the catalysts and occurs in a slurry media in which the pH is adjusted. Exemplary uses of the catalysts include packing for a methanation reactor that converts carbon monoxide and hydrogen into methane.

### CROSS-REFERENCE TO RELATED APPLICATIONS

This application is a non-provisional application which claims benefit under 35 USC §119(e) to U.S. Provisional Application Ser. No. 61/255,763 filed Oct. 28, 2009, entitled “CONTROLLING SYNTHESIS OF METAL SULFIDE CATALYSTS,” which is incorporated herein in its entirety.

### STATEMENT REGARDING FEDERALLY SPONSORED RESEARCH OR DEVELOPMENT

None

### FIELD OF THE INVENTION

Embodiments of the invention relate to metal sulfide.

### BACKGROUND OF THE INVENTION

Various compounds act as catalysts in applications to process feedstocks. Suitability of the catalysts depends on factors such as activity, preparation costs, susceptibility to deactivation, and selectivity for desired reactions. The catalysts can thus determine viability of processes such as methanation, which utilizes the catalysts to convert carbon monoxide and hydrogen gasses into methane.

Methanation reactions enable producing substitute natural gas (SNG) from coal by gasifying the coal to generate synthesis gas (syngas) and converting the syngas to the SNG via the methanation. Declining supplies of natural gas being produced and expanding demand for the natural gas cause natural gas prices to rise. Since coal resources are often more readily available than natural gas resources, the methanation provides an option for distributing these available energy sources as needed.

Prior compounds and preparation techniques for the compounds used as the catalysts in the methanation reactions impose operating limitations or prevent economic feasibility. Sulfur poisons nickel based compounds used for the catalyst in the methanation such that sulfur impurities from the feedstock must be removed within tolerances of the nickel based compounds. In addition, utilizing the nickel based compounds requires adjusting a hydrogen/carbon monoxide ratio since carbon deactivates the nickel based compounds. While prior molybdenum disulfide products when used as the catalyst for the methanation may lack such restrictions with respect to carbon and sulfur feedstock content, previous methods produce the molybdenum disulfide products with limited performance.

Therefore, a need exists for improved metal sulfide catalysts and methods of preparing such catalysts.

### SUMMARY OF THE INVENTION

In one embodiment, a method includes preparing a mixture comprising ions containing sulfur and metal. Adding an acid to the mixture results in a slurry containing precipitate of metal sulfide particles that are formed from the ions. The metal sulfide particles include at least one of molybdenum, tungsten and vanadium since a salt thereof provides the metal within the mixture. Precipitation progresses due to addition of the acid within the slurry over time. The precipitation progresses while preventing fluctuation in pH of the slurry of more than 1.0.

According to one embodiment, a method includes dissolving ammonium tetrathiomolybdate and zirconyl nitrate hydrate to provide a mixture. Adding an acid to the mixture results in a slurry containing a precipitate. Precipitation progresses by the acid being added within the slurry over time. The method further includes maintaining pH of the slurry above 4.0 by partly neutralizing overall acidity buildup as the precipitation progresses.

For one embodiment, a method includes preparing a mixture comprising ions containing sulfur and metal. Blending an acid and the mixture forms a resulting slurry while adjusting flow rate of at least one of the mixture and the acid being blended at least partly prevents acidification of the slurry increasing with addition of the acid. The slurry contains precipitated metal sulfide particles, which include at least one of molybdenum, tungsten and vanadium since a salt thereof provides the metal within the mixture. Contacting carbon monoxide and hydrogen with a catalyst of the metal sulfide particles produces methane.

### BRIEF DESCRIPTION OF THE DRAWINGS

The invention, together with further advantages thereof, may best be understood by reference to the following description taken in conjunction with the accompanying drawings.

FIG. 1 is a flow chart illustrating a method of preparing molybdenum disulfide catalyst, according to one embodiment.

FIG. 2 is a graph showing influence of pH during precipitation on carbon monoxide conversion using catalysts formed with precipitate resulting from the precipitation, according to one embodiment.

### DETAILED DESCRIPTION OF THE INVENTION

Embodiments of the invention relate to catalysts and preparation of the catalysts, which are defined by sulfides of a transition metal, such as one of molybdenum, tungsten, and vanadium. Precipitation forms the catalysts and occurs in a slurry media in which the pH is adjusted. Exemplary uses of the catalysts include packing for a methanation reactor that converts carbon monoxide and hydrogen into methane.

Precursors for the catalysts include a metal ion source compound, which may be a molybdenum salt, such as ammonium tetrathiomolybdate, an oxide of molybdenum (e.g., molybdenum trioxide), molybdenum sulfide (lacking activity as high as products described herein), molybdenum pentachloride, and ammonium heptamolybdate. While particular reference is made to molybdenum and preparation of molybdenum disulfide catalysts, methods described herein may instead utilize salts of other transition metals, such as vanadium and tungsten in place of molybdenum. Utilizing the ammonium thiomolybdate or the molybdenum sulfide as the metal ion source compound provides ions containing sulfur and molybdenum within a mixture. If not provided by the metal ion source compound, other sources of sulfur ions in the mixture may include hydrogen sulfide, sodium hydrosulfide, and sodium sulfide.

All precursor compounds are liquid or dissolvable in a liquid solvent. Once the precursors are dissolved if not liquid and combined in the mixture, adding any acid to the mixture may result in a slurry containing precipitate products. Suitability for the acid utilized depends on ability and ease in removing ions, such as nitrates from exemplary nitric acid used as the acid. Precipitation occurs with mixing of the slurry and adding of the acid over time. As shown herein, adjusting the pH of the slurry as the acid is added influences performance of the catalyst that is produced.

The catalysts may further include promoters, such as at least one of carbon, silicon, aluminum, zirconium, magnesium, potassium, palladium, titanium, cobalt, nickel and iron. During the precipitation, the mixture thus may contain ions of elements desired for the promoter such that co-precipitation may incorporate the promoter in the catalysts. For example, the mixture may include metal promoter salts that are dissolved.

Collecting of the precipitate that provides the molybdenum disulfide catalyst refers to any handling and treatment of the precipitate upon causing precipitation within the mixture. During further preparing of the catalyst, pelletizing the precipitate after being filtered, washed and dried shapes the precipitate into solid particles. In some embodiments, presulfiding and/or reduction adjusts ratio of molybdenum to sulfur within the precipitate and may promote creating a desirable form of molybdenum disulfide (MoS₂).

In some embodiments, the mixture that is created contains dissolved solutes including the ammonium tetrathiomolybdate (NH₄)₂MoS₄. The solutes for some embodiments further include a metal promoter, such as a zirconium-containing compound that may be a zirconium salt (e.g., zirconyl nitrate hydrate (H₂N₂O₈Zr)). One or more suitable solvents enable dissolution of the solutes for forming the mixture, which with the ammonium tetrathiomolybdate may be aqueous such that addition of water controls dilution of the solutes to desired levels for mixing together and subsequent co-precipitation. In some embodiments, acids and/or bases facilitate in dissolving the solutes.

For some embodiments, preparation of the mixture includes making a first solution with the ammonium tetrathiomolybdate and combining the first solution with a second solution of the zirconyl nitrate hydrate. The first solution that is basic mixes with the second solution that is acidic in quantities such that the mixture has a pH greater than is desired to maintain the pH of the slurry during the precipitation. Compared to the acid added to the mixture to cause the precipitation, a smaller volume of the second solution mixes with the first solution over sufficient time so as to not function as a precipitating agent. The acid added to the mixture to cause the precipitation tends to reduce the pH of the slurry since the acid has a lower pH than is desired to maintain the pH of the slurry during the precipitation. Controlling flow rates of the mixture and the acid can thereby maintain the pH of the slurry as desired.

The controlling of the flow rates maintains the pH of the slurry in some embodiments above about 4.0, between about 4.0 and about 6.0, or between about 5.0 and about 6.0. Such maintaining of the pH prevents fluctuations in the pH of more than about 1.5, about 1.0 or about 0.5, in some embodiments. Conducting the precipitation with pH adjustment to certain threshold values facilitates synthesis since the threshold values rely on quantitative techniques without depending on qualitative criteria such as visual inspection. While not limited to any particular theory, at least one effect of adjusting the pH of the slurry as set forth herein includes altering a promoter metal (e.g., zirconium) to molybdenum mol ratio in the precipitate that is formed. The pH of the slurry in combination with amount of zirconium present in the mixture achieves the zirconium to molybdenum mol ratio of between about 0.5 and about 1.0 or between about 0.6 and about 0.8, for some embodiments. Adjusting the pH of the slurry as set forth herein may further yield the precipitate with a higher surface area and smaller crystal domain size than if the pH is not maintained and hence fluctuates more than 1.0.

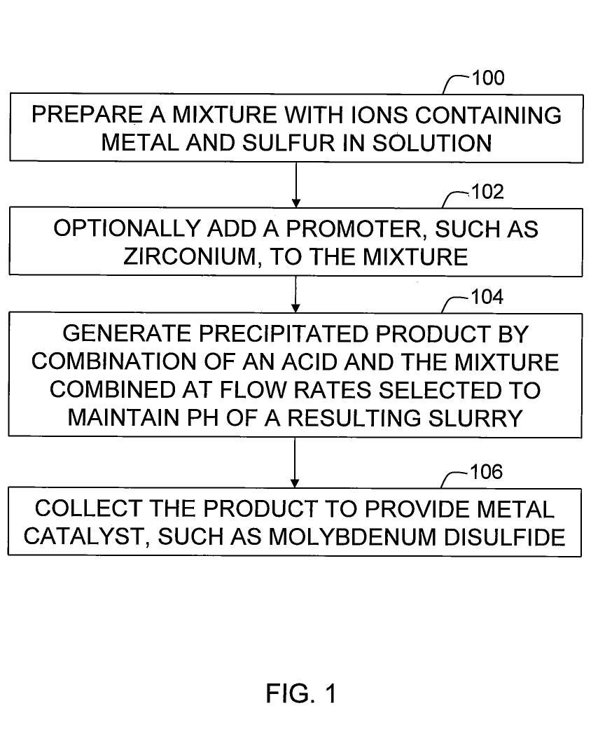

FIG. 1 illustrates a flow chart for an exemplary method of preparing molybdenum disulfide catalyst. In a dissolution step 100, preparing a solution of ions that contain metal and sulfur provides a mixture, which may be formed of ammonium tetrathiomolybdate dissolved in water. For some embodiments, a promoter addition step 102 introduces a promoter compound into the mixture. During subsequent precipitation, the promoter compound may result in incorporating another metal in addition to molybdenum into the molybdenum disulfide catalyst. Utilizing zirconyl nitrate hydrate as the promoter thereby makes the molybdenum disulfide catalyst contain zirconium (e.g., zirconium dioxide (ZrO₂)). Adding the promoter may occur by blending with the mixture at a pH that may be basic an amount of zirconyl nitrate hydrate dissolved in water at a pH that may be acidic. A resulting pH (e.g, pH range between 6 and 7) of the mixture after adding the zirconyl nitrate hydrate ensures that the zirconyl nitrate hydrate is or stays dissolved when mixed with the ammonium tetrathiomolybdate that also stays dissolved.

Precipitation step 104 occurs by addition of an acid, such as nitric acid, to the mixture. The mixture and the nitric acid combine over a time interval. During the time interval, flow rates for the mixture and the nitric acid maintain pH of a slurry formed. The slurry contains solid precipitate. Precipitate recovery step 106 separates and collects the solid precipitate by filtration. The precipitate collected in the recovery step 106 provides the molybdenum disulfide catalyst.

In some embodiments, the precipitate recovery step 106 includes filtering the mixture to isolate the precipitate. Next, washing the precipitate with water and then a volatile fluid, such as acetone or ethanol, removes unwanted impurities and water from the precipitate. The precipitate after being washed undergoes drying under an inert or nitrogen atmosphere at a temperature (e.g., between 50° C. and 150° C.) above ambient to facilitate evaporation for a duration of at least three hours, or until the precipitate has a constant weight. Once dried, the precipitate can be made into tablets by compression in a metal die to form cylindrical pellets or otherwise shaped to form catalyst particles.

In some embodiments, treating the precipitate with elemental sulfur enhances stability of the catalyst particles. For example, the treating may include mixing the precipitate, prior to drying the precipitate, in a suspension of the elemental sulfur dispersed in a liquid, such as acetone. The precipitate and elemental sulfur together then undergo drying and further collecting as described herein.

While not limited to any particular theory, it is believed that the precipitate may include molybdenum trisulfide (MoS₃) prior to any reduction processing of the precipitate. The molybdenum trisulfide thus becomes an intermediate to formation of the molybdenum disulfide. Reducing the pellets of the precipitate converts the molybdenum trisulfide into the molybdenum disulfide. Presulfiding the pellets of the precipitate may occur in addition to the reducing to ensure this conversion proceeds without over reduction of molybdenum disulfide. During the presulfiding, the pellets of the precipitate contact a flow of a sulfur-containing fluid, such as dimethyl disulfide (C₂H₆S₂) or hydrogen sulfide (H₂S), while disposed within a reactor heated to above 300° C. The reduction of the precipitate includes exposing the precipitate to a reducing environment. Temperatures above 300° C. or between 400° C. and 700° C. along with contact of the precipitate with a hydrogen-containing gas define conditions for the reducing environment. In combination with hydrogen gas, the hydrogen-containing gas may include hydrogen sulfide and dimethyl disulfide to prevent over reduction of the molybdenum disulfide catalyst.

### Examples

A first solution was prepared by dissolving 8 grams of ammonium tetrathiomolybdate in 123.04 milliliters (ml) of de-ionized (DI) water. The first solution was mixed with a second solution having 3.552 grams of zirconyl nitrate hydrate dissolved in 8.752 ml of DI water. Precipitation was done by acidification with 0.1 molar (M) nitric acid added to a salt mixture of the first and second solutions. Respective pumps supplied the nitric acid and the salt mixture at flow rates controlled to maintain pH obtained upon mixing the nitric acid the salt mixture at 3 (Example 1) throughout synthesis of precipitate. Separate samples were prepared and analyzed analogous to Example 1 described herein except that the pH was maintained at 4, 5 and 6 for respective Examples 2, 3 and 4.

The precipitate was then filtered, washed with distilled water first and then washed with acetone. A resultant cake was stirred in acetone with 3.076 grams of sulfur prior to being dried in an oven under nitrogen atmosphere at 80° C. for 4 hours and then pelletized to form precipitate pellets.

The precipitate pellets were pre-sulfided and reduced. A reactor was loaded with 3.8 ml of the pellets mixed with 6.2 ml of alundum. The reactor was heated to 450° C. at 3171 kilopascal with nitrogen flowing through the reactor at a rate of 45 cubic centimeters per minute (cc/min) along with dimethyl disulfide (DMDS) flowing at 0.1 ml per hour (ml/hr). When the temperature reached 450° C., flow of the nitrogen was switched to a stream of hydrogen at a 45.5 cc/min flow rate along with DMDS flowing at 0.15 ml/hr. The reactor was kept at 500° C. under hydrogen and DMDS for 5 hours in order to provide a sample molybdenum disulfide catalyst.

A fixed bed activity test was performed on the sample molybdenum disulfide catalyst. For the activity test, reactor pressure was 3171 kilopascal (kPa) with a gas hourly space velocity (GHSV) of 2400 hr⁻¹ and hydrogen to carbon monoxide ratio (H₂/CO) of 1.08. Reactor temperature was set at 455° C. A feed gas stream was about 37% H₂, 34% CO, 1% H₂S and 28% N₂, which substituted for other inert gasses. The hydrogen to carbon ratio of 1.08 used for the test illustrated suitability of the catalyst for coal derived syngas without having to adjust the ratio to higher values with water gas shift reactions. The hydrogen sulfide within the feed gas stream simulated sulfur impurities common in syngas and that can poison prior catalysts. Such sulfur content illustrates ability to process syngas in which sulfur impurities are removed to meet requirements (e.g., 4 parts per million sulfur) for natural gas pipeline transport of produced methane. No need exists for expensive sulfur removal to limitations (e.g., 20 parts per billion) imposed by some prior catalysts (i.e., nickel based materials).

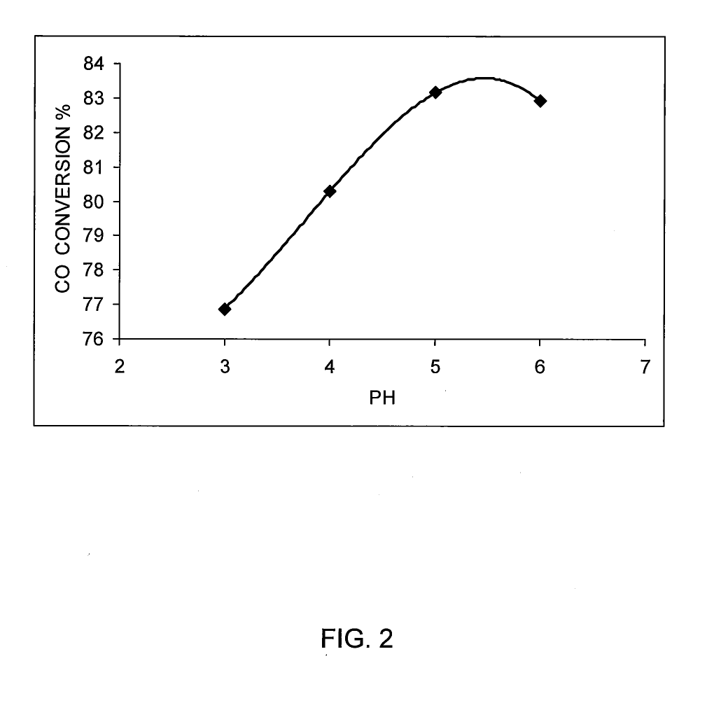

FIG. 2 shows results of the activity test that were determined for each of the Examples 1-4 and an anticipated trend line. Carbon monoxide conversion increased as a function of raising the pH that the slurry was kept up to pH of 5. The carbon monoxide conversion obtained was about 83% when the pH of the slurry was maintained at 5. Given the carbon to hydrogen ratio, substantially no water was produced along with main products defined by carbon dioxide and methane. Methane selectivity, as defined by relative conversion of the carbon monoxide into methane compared to carbon dioxide, of about 52% resulted based on methane and carbon dioxide yields.

A comparative catalyst was also prepared. For the comparative catalyst, a salt solution was prepared by mixing two aqueous solutions—4 grams of ammonium tetrathiomolybdate in 61.52 ml of water, and 1.776 grams of zirconyl nitrate hydrate in 6 ml of water. The salt solution was further acidified with diluted nitric acid but without taking any action to maintain pH of a resulting slurry. The resulting slurry was filtered and washed with distilled water and acetone. A resultant cake was added to 1.538 grams of sulfur stirred in acetone. After removing excess acetone, the resultant cake was dried, pre-sulfided and reduced as described herein with respect to the Example 1. The activity test as set forth herein was conducted again using the comparative sample. While the molybdenum disulfide catalyst prepared in accordance with Example 3 for instance provided about 83% conversion of carbon monoxide, the comparative sample only resulted in about 74% conversion of carbon monoxide.

The preferred embodiment of the present invention has been disclosed and illustrated. However, the invention is intended to be as broad as defined in the claims below. Those skilled in the art may be able to study the preferred embodiments and identify other ways to practice the invention that are not exactly as described herein. It is the intent of the inventors that variations and equivalents of the invention are within the scope of the claims below and the description, abstract and drawings are not to be used to limit the scope of the invention.

What is claimed is:

1. A method comprising:
preparing a mixture comprising ions of sulfur and metal;
adding an acid to the mixture results in a slurry containing precipitate of metal sulfide particles formed from the ions, wherein the metal sulfide particles include at least one of molybdenum, tungsten and vanadium since a salt thereof provides the metal within the mixture; and
preventing fluctuation in pH of the slurry of more than 1.0 as precipitation progresses, wherein the precipitation progresses due to the adding of the acid within the slurry over time.

2. The method according to claim 1, wherein the salt is a molybdenum salt.

3. The method according to claim 1, wherein the salt is ammonium tetrathiomolybdate.

4. The method according to claim 1, wherein the mixture continues to be introduced within the slurry as the precipitation progresses.

5. The method according to claim 1, wherein the preventing fluctuation in the pH is based on flow rates in which the acid and the mixture are introduced together.

6. The method according to claim 1, wherein the mixture further includes zirconium ions from a zirconium salt such that a zirconium to molybdenum mol ratio in the metal sulfide particles is between 0.6 and 0.8.

7. The method according to claim 1, wherein the mixture further includes zirconium ions from zirconyl nitrate hydrate.

8. The method according to claim 1, wherein the acid is nitric acid, the salt is ammonium tetrathiomolybdate and the mixture further includes zirconium ions from zirconyl nitrate hydrate.

9. The method according to claim 1, further comprising shaping the metal sulfide particles into catalyst material.

10. The method according to claim 1, further comprising loading the metal sulfide particles into a methanation reactor that converts carbon monoxide and hydrogen into methane.

11. The method according to claim 1, further comprising contacting carbon monoxide and hydrogen with a catalyst of the metal sulfide particles to produce methane.

12. The method according to claim 1, wherein the preventing fluctuation in the pH maintains the pH of the slurry between 5 and 6 throughout the adding of the acid.

13. A method comprising:
dissolving ammonium tetrathiomolybdate and zirconyl nitrate hydrate to provide a mixture;
adding an acid to the mixture results in a slurry containing a precipitate; and
maintaining pH of the slurry above 4.0 by partly neutralizing overall acidity buildup as precipitation progresses, wherein the precipitation progresses by the acid being added within the slurry over time.

14. The method according to claim 13, wherein the acid includes nitric acid.

15. The method according to claim 13, wherein the pH of the mixture is above 4.0, the pH of the acid is below 4.0, and the maintaining of the pH is based on respective flow rates in which the acid and the mixture are introduced together for forming the slurry.

16. The method according to claim 13, wherein the maintaining of the pH keeps the pH of the slurry between 5 and 6 throughout the adding of the acid.

17. The method according to claim 13, further comprising loading the molybdenum disulfide catalyst into a methanation reactor that converts carbon monoxide and hydrogen into methane.

18. A method comprising:
preparing a mixture comprising ions containing sulfur and metal;
blending an acid and the mixture to form a resulting slurry while adjusting flow rate of at least one of the mixture and the acid being blended at least partly prevents acidification of the slurry increasing with addition of the acid, wherein the slurry contains precipitated metal sulfide particles, which include at least one of molybdenum, tungsten and vanadium since a salt thereof provides the metal within the mixture; and
contacting carbon monoxide and hydrogen with a catalyst of the metal sulfide particles to produce methane.

19. The method according to claim 18, wherein the metal sulfide particles further include zirconium from zirconyl nitrate hydrate added to the mixture.

20. The method according to claim 18, wherein the flow rates maintain the pH of the slurry between 4 and 6 throughout the blending of the acid with the mixture.
